(19) United States
(12) Patent Application Publication
(10) Pub. No.: US 2025/0050066 A1
(43) Pub. Date: Feb. 13, 2025

(54) MULTI-NEEDLE INTRATUMORAL INJECTION INSTRUMENT

(71) Applicant: Covidien LP, Mansfield, MA (US)

(72) Inventors: Jennifer S. Mattingley, La Crosse, WI (US); Scott E. M. Frushour, Boulder, CO (US); Daniel A. Ledeen, Austin, TX (US)

(21) Appl. No.: 18/740,737

(22) Filed: Jun. 12, 2024

Related U.S. Application Data
(60) Provisional application No. 63/517,949, filed on Aug. 7, 2023.

(51) Int. Cl. A61M 25/00; A61M 5/158; A61M 25/06; A61M 29/00

(52) U.S. Cl. CPC A61M 25/0084; A61M 5/158; A61M 29/00; A61M 2005/1581; A61M 2025/0087; A61M 25/0097; A61M 2025/0681; A61M 2205/0266

(57) ABSTRACT

A surgical instrument includes a supply chamber, a handle assembly, a sleeve extending from the handle assembly, a distal ring, a plurality of needle tines, and a deflector. The plurality of needle tines extends through a lumen of the sleeve, is movable through the lumen of the sleeve via the handle assembly, and is configured to dispense a therapeutic agent from the supply chamber. The deflector extends through the lumen and is movable through the lumen via the handle assembly. Retraction of the deflector relative to the sleeve and the plurality of needle tines causes the needle tines to deflect away from a central longitudinal axis of the sleeve against the distal ring to change an angle between a trajectory of the plurality of needle tines and the central longitudinal axis.

### CROSS-REFERENCE TO RELATED APPLICATIONS

This application claims the benefit of the filing date of provisional U.S. Patent Application No. 63/517,949 filed on Aug. 7, 2023.

### FIELD

This disclosure is generally related to surgical instruments and, more particularly, to surgical instruments including a plurality of injection needles for intratumoral injections.

### BACKGROUND

Clinical acceptance of intratumoral injection is becoming more prevalent in interventional medicine. Intratumoral injection includes accessing a tumor and injecting therapeutic agents with the idea of compartmentalizing the agent to a localized region to avoid systemic side effects that have historically been associated with chemotherapy and other treatment modalities.

In many solid tumors, the disease may present homogeneous across the mass.

However, in some tumors and particularly in the lung, the disease presents in a more heterogenous condition. This means that within a lung nodule, you may find both diseased and healthy cells. Because of this phenomena, it is advisable to sample tissue from various places within the nodule to affirm the nodule is indeed malignant.

To effectively treat the lesion with intratumoral injection it is desirable to ensure uniform diffusion of a therapy agent across the entire volume of the lesion. This is accomplished with a single needle that enters the lesion utilizing multiple capsular invasion points. The challenge with this concept is that these holes in the tumor capsule allow for the therapy agent to diffuse outside the boundary and into other systemic organs, thus reducing the effectiveness of the procedure.

### SUMMARY

This disclosure is directed to surgical instruments including a plurality of injection needles to uniformly dispense a therapeutic agent into different portions of a tumor for intratumoral injections.

In accordance with aspects of the disclosure, a surgical intratumoral injection instrument includes a supply chamber, a handle assembly, a sleeve extending from the handle assembly, a distal ring, a plurality of needle tines, and a deflector. The plurality of needle tines extends through a lumen of the sleeve, is movable through the lumen of the sleeve via the handle assembly, and is configured to dispense a therapeutic agent from the supply chamber. The deflector extends through the lumen and is movable through the lumen via the handle assembly. Retraction of the deflector relative to the sleeve and the plurality of needle tines causes the plurality of needle tines to deflect away from a central longitudinal axis of the sleeve against the distal ring to change an angle between a trajectory of the plurality of needle tines and the central longitudinal axis.

In an aspect, the deflector may include an anvil portion configured to abut the plurality of needle tines and apply a force to the plurality of needle tines against the distal ring.

In an aspect, distal advancement of the plurality of needle tines relative to the deflector a first distance may deflect the plurality of needle tines at a first angle relative to the central longitudinal axis, and distal advancement of the plurality of needle tines relative to the deflector a second distance may deflect the plurality of needle tines at a second angle relative to the central longitudinal axis.

In an aspect, retraction of the deflector relative to the sleeve and the plurality of needle tines a first distance may deflect the plurality of needle tines at a first angle relative to the central longitudinal axis, and retraction of the deflector relative to the sleeve and the plurality of needle tines a second distance may deflect the plurality of needle tines at a second angle relative to the central longitudinal axis.

In an aspect, a plurality of rounded grooves may be defined along a length of an outer surface of the deflector and each needle tine of the plurality of needle tines may be disposed within a respective rounded groove of the plurality of rounded grooves.

In an aspect, the plurality of needle tines may be formed of a shape memory material possessing a predefined curvature.

In an aspect, a distal end of the deflector may include a blunt dilator tip.

In an aspect, the handle assembly may include a first handle, a second handle, and a third handle. The first handle may be operably coupled to the plurality of needle tines and configured to control movement of the needle tines relative to the deflector. The second handle may be operably coupled to the sleeve and configured to control movement of the sleeve relative to the plurality of needle tines and the deflector. The third handle may be operably coupled to the deflector and configured to control movement of the deflector relative to the plurality of needle tines and the sleeve.

In an aspect, the sleeve may be formed of a flexible material and the distal ring may be formed of a rigid material.

In an aspect, each needle tine of the plurality of needle tines may be independently movable relative to other needle tines of the plurality of needle tines.

In another aspect of the disclosure, a surgical intratumoral injection system includes a guide catheter and a surgical intratumoral injection instrument. The guide catheter defines a lumen and is configured to be navigated to a target. The surgical intratumoral injection instrument is configured to be inserted through the lumen of the guide catheter to access the target. The surgical intratumoral injection instrument includes a supply chamber, a handle assembly, a sleeve extending from the handle assembly, a distal ring, a plurality of needle tines, and a deflector. The plurality of needle tines extends through a lumen of the sleeve, is movable through the lumen of the sleeve via the handle assembly, and is configured to dispense a therapeutic agent from the supply chamber. The deflector extends through the lumen and is movable through the lumen via the handle assembly. Retraction of the deflector relative to the sleeve and the plurality of needle tines causes the plurality of needle tines to deflect away from a central longitudinal axis of the sleeve against the distal ring to change an angle between a trajectory of the plurality of needle tines and the central longitudinal axis.

In an aspect, the deflector may include an anvil portion configured to abut the plurality of needle tines and apply a force to the plurality of needle tines against the distal ring.

In an aspect, distal advancement of the plurality of needle tines relative to the deflector a first distance may deflect the plurality of needle tines at a first angle relative to the central longitudinal axis, and distal advancement of the plurality of needle tines relative to the deflector a second distance may deflect the plurality of needle tines at a second angle relative to the central longitudinal axis.

In an aspect, retraction of the deflector relative to the sleeve and the plurality of needle tines a first distance may deflect the plurality of needle tines at a first angle relative to the central longitudinal axis, and retraction of the deflector relative to the sleeve and the plurality of needle tines a second distance may deflect the plurality of needle tines at a second angle relative to the central longitudinal axis.

In an aspect, a plurality of rounded grooves may be defined along a length of an outer surface of the deflector and each needle tine of the plurality of needle tines may be disposed within a respective rounded groove of the plurality of rounded grooves.

In an aspect, the plurality of needle tines may be formed of a shape memory material possessing a predefined curvature.

In an aspect, a distal end of the deflector may include a blunt dilator tip.

In an aspect, the handle assembly may include a first handle, a second handle, and a third handle. The first handle may be operably coupled to the plurality of needle tines and configured to control movement of the needle tines relative to the deflector. The second handle may be operably coupled to the sleeve and configured to control movement of the sleeve relative to the plurality of needle tines and the deflector. The third handle may be operably coupled to the deflector and configured to control movement of the deflector relative to the plurality of needle tines and the sleeve.

In an aspect, the sleeve may be formed of a flexible material and the distal ring may be formed of a rigid material.

In an aspect, each needle tine of the plurality of needle tines may be independently movable relative to other needle tines of the plurality of needle tines.

In another aspect of the disclosure, a surgical intratumoral injection instrument includes a supply chamber, a handle assembly, a sleeve, and a plurality of needle tines configured to dispense a therapeutic agent from the supply chamber to a tumor. The sleeve extends from the handle assembly and defines a lumen. The plurality of needle tines extends through the lumen of the sleeve and is movable through the lumen of the sleeve via the handle assembly. The plurality of needle tines is formed of a shape memory material configured to splay radially outward from a central longitudinal axis of the sleeve when the plurality of needle tines is deployed from the sleeve and configured to curve inward toward the central longitudinal axis of the sleeve as the plurality of needle tines is inserted into the tumor.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects of the disclosure are described herein below with reference to the drawings, wherein:

FIG. 1 is a side view of a surgical injection instrument in accordance with the disclosure;

FIG. 2 is a side cross-sectional view of a distal portion of the surgical injection instrument of FIG. 1 with the surgical injection instrument in a first condition;

FIG. 3 is a side cross-sectional view of a distal portion of the surgical injection instrument of FIG. 1 with the surgical injection instrument in a second condition;

FIG. 4 is a side cross-sectional view of a distal portion of the surgical injection instrument of FIG. 1 with the surgical injection instrument in a third condition;

FIG. 5 is a front cross-sectional view of a portion of the surgical injection instrument of FIG. 1 in accordance with an aspect of the disclosure;

FIG. 6A is a side perspective view of an example of needle tines of the surgical injection instrument of FIG. 1 in accordance with an aspect of the disclosure;

FIG. 6B is a side perspective view of an example of needle tines of the surgical injection instrument of FIG. 1 in accordance with another aspect of the disclosure; and

FIG. 6C is a side perspective view of an example of needle tines of the surgical injection instrument of FIG. 1 in accordance with another aspect of the disclosure.

### DETAILED DESCRIPTION

In this description, the term “proximal” is used generally to refer to that portion of the device that is closer to a clinician, while the term “distal” is used generally to refer to that portion of the device that is farther from the clinician. Further, the term “clinician” is used generally to refer to medical personnel including doctors, nurses, and support personnel or any other user of the disclosed instruments.

The disclosure describes a multi-needle injection instrument that is configured for intratumoral injection of therapeutic fluids to a target (e.g., tumor). The disclosed surgical intratumoral injection instrument 200 is designed in a manner that can be operated with one hand, injects a therapeutic agent at predetermined volumes for each actuation of an actuator, and can be manipulated to uniformly inject the therapeutic agent into different regions of a target from a single access/entry point into the target, that is, without the need to remove and reinsert the instrument into the target multiple times.

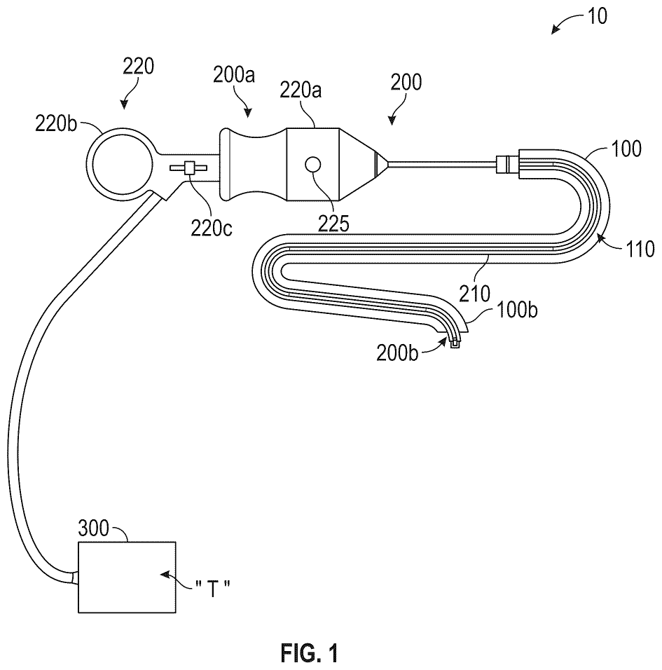

FIG. 1 illustrates a surgical intratumoral injection system 10 including a guide catheter 100, an intratumoral injection instrument 200, and a supply chamber 300 configured to store a therapeutic agent “T”. Although the supply chamber 300 is illustrated as external to the intratumoral injection instrument 200, in aspects, the supply chamber 300 may be an internal component of the intratumoral injection instrument 200.

The guide catheter 100 may be flexible or rigid and is configured to be navigated to a target site within a patient and to act as a conduit for the intratumoral injection instrument 200 to access a target (e.g., tumor) located within the target site. The guide catheter 100 may be navigated manually or robotically to the target site using any suitable navigation system, for example using an electromagnetic navigation system. Either or both of the guide catheter 100 and the intratumoral injection instrument 200 may be handheld devices or devices configured to interface with a robotic arm of a surgical robotic system. Upon placement of the distal end 100b of the guide catheter 100 proximate the target site, the intratumoral injection instrument 200 may be inserted through a lumen 110 of the guide catheter 100 such that a distal portion 200b of the intratumoral injection instrument 200 protrudes from the distal end 100b of the guide catheter 100 and into the target.

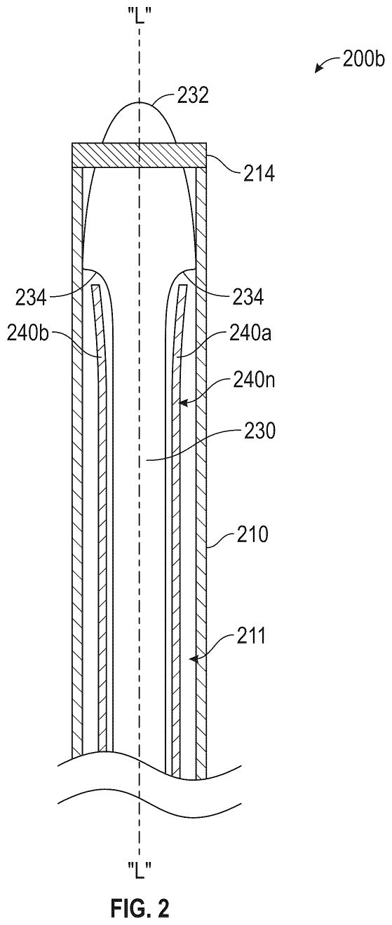

With continued reference to FIG. 1 and with additional reference to FIGS. 2-4, the intratumoral injection instrument 200 includes a handle assembly 220 at its proximal portion 200a and a sleeve 210, which defines a lumen 211, extending distally from the handle assembly 220. The sleeve 210 may be flexible or rigid and includes a distal ring 214 formed of a rigid material at its distal end. The intratumoral injection instrument 200 also includes a plurality of needle tines 240n (referred to individually as needle tines 240a, 240b . . . n) and a deflector 230 extending through the lumen 211 of the sleeve 210 from the handle assembly 220 along a central longitudinal axis “L” of the sleeve 210.

The needle tines 240a, 240b . . . n (referred to collectively as the plurality of needle tines 240n) are movable through the lumen 211 of the sleeve 210 via the handle assembly 220 and are configured to dispense the therapeutic agent “T” from the supply chamber 300 into the target via actuation of an actuator 225. In aspects of the present disclosure, any one or more of the plurality of needle tines 240n may be serve as a supply chamber configured to store the therapeutic agent “T” either in lieu of the supply chamber 300 or in conjunction with the supply chamber 300.

In an aspect, the plurality of needle tines 240n is coupled to a first handle 220a of the handle assembly 220 to control longitudinal movement of the plurality of needle tines 240n relative to at least one of the deflector 230 or the sleeve 210 along the central longitudinal axis “L”. In aspects, the first handle 220a of the handle assembly 220 includes individual selectable connections to each needle tine 240a, 240b . . . n of the plurality of needle tines 240n and is configured to control individual movement of each needle tine 240a, 240b . . . n relative to the others when selected for movement. In aspects of the present disclosure, movement of each individual needle tine 240a, 240b . . . n may be controlled manually by the clinician via the handle assembly 220 and/or controlled robotically by a robotic arm of a surgical robotic system. For example, the handle assembly 220 may be configured to interface with a robotic arm of a surgical robotic system to facilitate robotic control (e.g., deployment, retraction, manipulation, positioning, placement, etc.) of each individual needle tine of the plurality of needle tines 240n. By way of another example, one or more needle tines of the plurality of needle tines 240n may be configured to interface directly with a robotic arm of a surgical robotic system to facilitate direct robotic control of each individual needle tine of the plurality of needle tines 240n. Additionally, the first handle 220a, or another portion of the handle assembly 220, may include a lock component (not shown) to lock the longitudinal position of one or more of the plurality of needle tines 240n along the central longitudinal axis “L”. The actuator 225 may be any suitable device or combination of devices, such as a valve, pump, and/or switch capable of initiating the delivery of the therapeutic agent “T” from the supply chamber 300 to the plurality of needle tines 240n for dispensing out of the plurality of needle tines 240n and into the target.

The plurality of needle tines 240n may be formed of alloys (e.g., nitinol, stainless steel, titanium, etc.) that allow for flexibility and possess shape memory characteristics. In aspects, the plurality of needle tines 240n are designed to be self-expanding and may be formed to have a preset curvature that causes the plurality of needle tines 240n to blossom from the central longitudinal axis “L” to an angle that folds back in a retrograde fashion. Such a configuration results in maximum angulation of the trajectory of the plurality of needle tines 240n relative to the central longitudinal axis “L”.

In an aspect, the deflector 230 includes a dilator tip 232 at its distal end which may be blunt to prevent atraumatic injuries to the target or tissue proximate the target within the target site during movement of the deflector 230 or navigation of the intratumoral injection instrument 200. In other aspects, the deflector 230 includes a dilator tip 232 at its distal end that is needle-like (e.g., pointed) to enable the percutaneous insertion of the deflector 230 and/or the plurality of needle tines 240n into the target. A portion of the deflector 230 includes an anvil portion 234 that depresses against the plurality of needle tines 240n as the deflector 230 is retracted proximally relative to the plurality of needle tines 240n and/or which the plurality of needle tines 240n can slide along as the plurality of needle tines 240n is advanced distally relative to the deflector 230. Depression of the anvil portion 234 against the plurality of needle tines 240n (during proximal retraction of the deflector 230) urges the plurality of needle tines 240n against the distal ring 214 to counter-deflect the plurality of needle tines 240n. In aspects, the distal ring 214 includes radius edge to minimize acute pressure and friction against the plurality of needle tines 240n. Additionally, in some aspects, the distal ring 214 includes a beveled or chamfered edge to further define the deployment angle of the plurality of needle tines 240n as the deflector 230 is proximally retracted.

FIG. 2 illustrates the distal portion 200b of the intratumoral injection instrument 200 in a condition (e.g., a first condition) for insertion through the lumen 110 of the guide catheter 100. In this condition, the deflector 230 and the plurality of needle tines 240n are in their fully retracted positions. In an aspect, and as illustrated in FIG. 2, when the deflector 230 is fully retracted, a portion of the dilator tip 232 protrudes from the lumen 211 of the sleeve 210 to aid in navigation of the intratumoral injection instrument 200 to the target site. Also, when the plurality of needle tines 240n is fully retracted, the distal end of each needle tine 240a, 240b . . . n, is within the lumen 211 of the sleeve 210, thereby reducing the risk of injury to anatomical structures within the target site during movement of the intratumoral injection instrument 200 within the target site and/or to other anatomical structures during navigation of the intratumoral injection instrument 200 to the target site. The intratumoral injection instrument 200 may be visible under imaging guidance (e.g., fluoroscopic, white-light, ultrasound, cone-beam computed tomography (“CBCT”), etc.) to visualize the placement of the distal portion 200b relative to the target and to visualize the deployment trajectories of the plurality of needle tines 240n within the target throughout the procedure.

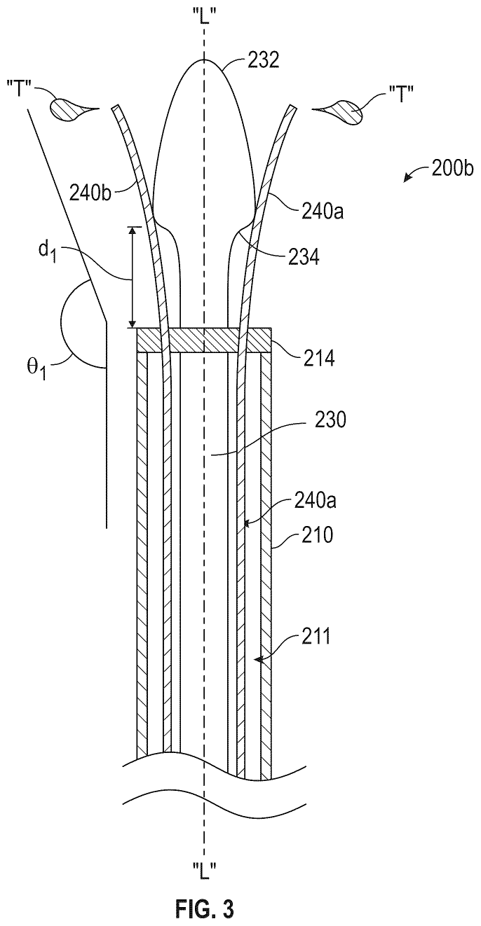

FIG. 3 illustrates the distal portion 200b of the intratumoral injection instrument 200 in a condition (e.g., a second condition) for a first delivery of the therapeutic agent “T” to the target. In particular, after the distal portion 200b of the intratumoral injection instrument 200 is navigated to the target, the plurality of needle tines 240n is advanced distally (e.g., via manipulation of the first handle 220a of the handle assembly 220) to extend closer to, or beyond, the distal end of the deflector 230. In addition to distally advancing the plurality of needle tines 240n, either the sleeve 210 is retracted proximally (e.g., via manipulation of the second handle 220b of the handle assembly 220) or the deflector 230 is distally advanced (e.g., via manipulation of the third handle 220c of the handle assembly 220) resulting in both the deflector 230 and the plurality of needle tines 240n being placed in the target with a distance d1 between the anvil portion 234 of the deflector 230 and the distal end of the distal ring 214. In this condition, a first deployment angle θ1 (e.g., obtuse) is formed between a trajectory of the plurality of needle tines 240n and the central longitudinal axis “L” and the therapeutic agent “T” is uniformly dispensed into the target from the plurality of needle tines 240n at the first deployment angle θ1 via actuation of the actuator 225.

After the therapeutic agent “T” is uniformly dispensed into the target from the plurality of needle tines 240n with the plurality of needle tines 240n positioned at the first deployment angle θ1, the deployment angle of the plurality of needle tines 240n may be changed to a second deployment angle θ2 for uniform distribution of the therapeutic agent “T” from the plurality of needle tines 240n to another region of the target without removing the distal portion 200b from the target.

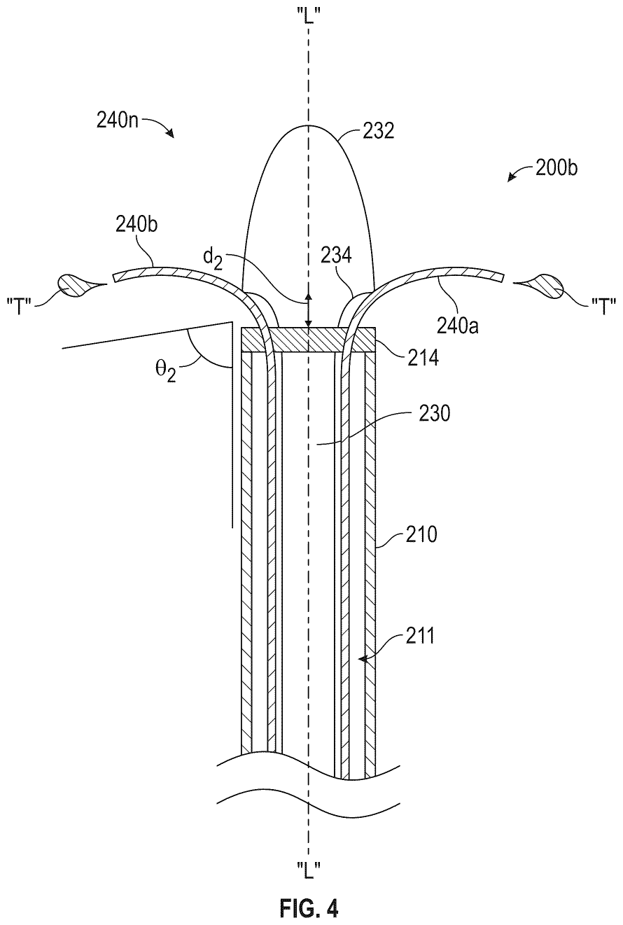

In particular, FIG. 4 illustrates the intratumoral injection instrument 200 in a condition (e.g., a third condition) for a second delivery of the therapeutic agent “T” to the target with the plurality of needle tines 240n positioned at a second deployment angle θ2 (e.g., acute) relative to the central longitudinal axis “L”. In order to change the deployment angle from θ1 to θ2, the deflector 230 is retracted proximally (e.g., via manipulation of the third handle 220c of the handle assembly 220) while the sleeve 210 and the plurality of needle tines 240n are fixed in their longitudinal positions. In particular, the deflector 230 is retracted proximally to create a smaller distance d2 between the anvil portion 234 of the deflector 230 and the distal end of the distal ring 214. As the deflector 230 is retracted proximally, the anvil portion 234 depresses the outer surface of each needle tine 240a, 240b . . . n, forcing the plurality of needle tines 240n to deflect radially out away from the central longitudinal axis “L” to create the deployment angle θ2 between the trajectory of the plurality of needle tines 240n and the central longitudinal axis “L”.

In aspects of the present disclosure, distal advancement and proximal retraction of the deflector 230 and/or the sleeve 210 may be controlled manually by the clinician via the handle assembly 220 and/or controlled roboticially by a robotic arm of a surgical robotic system. For example, the handle assembly 220 may be configured to interface with a robotic arm of a surgical robotic system to facilitate robotic control of the deflector 230 including the anvil portion 234 and/or to facilitate robotic control of the sleeve 210. By way of another example, the deflector 230 and/or the sleeve 210 may be configured to interface directly with a robotic arm of a surgical robotic system to facilitate direct robotic control of the deflector 230 including the anvil portion 234 and/or to facilitate direct robotic control of the sleeve 210.

While two deployment angles are described above, the deflector 230 and/or plurality of needle tines 240n may be further manipulated to achieve more than two deployment angles for additional uniform injection of the therapeutic agent “T” to different regions of the target, as a clinician desires for a given target and patient. Additionally, each needle tine 240a, 240b . . . n may be selected to be independently controlled and deployed by the first handle 220a (or another component of the handle assembly 220) such that only one needle tine or some needle tines of the plurality of needle tines 240n is moved to a different portion of the target upon movement of the first handle 220a (or other component of the handle assembly 220). In aspects, the actuator 225 may also be manipulated to select one or more specific needle tines 240a, 240b . . . n of the plurality of needle tines 240n through which the therapeutic agent “T” will be delivered.

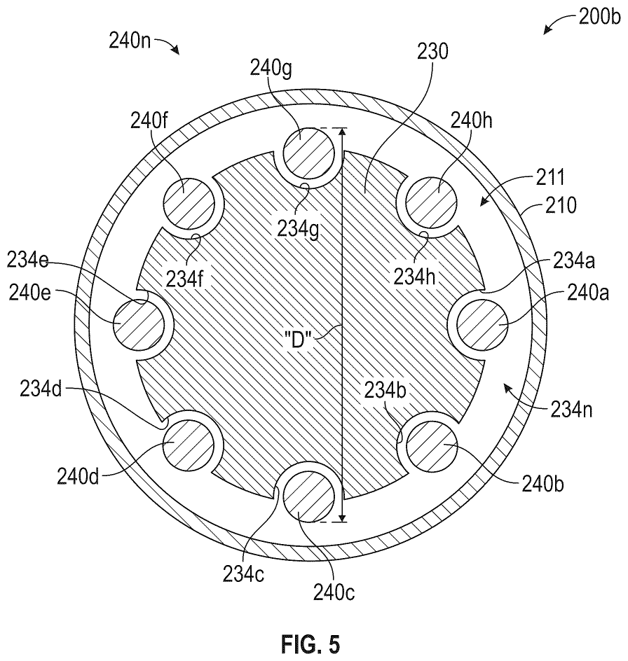

FIG. 5 illustrates an aspect of a deflector 230 including rounded grooves 234a . . . h (referred to collectively as a plurality of rounded grooves 234n) formed along a length of an outer surface of the deflector 230. One needle tine 240a . . . h of the plurality of needle tines 240n is seated within a respective rounded groove 234a . . . h of the plurality of rounded grooves 234n, thereby reducing the overall combined diameter “D” of the plurality of needle tines 240n/deflector 230 combination. A reduced overall combined diameter “D” enables the use of a smaller sleeve 210 for fitment within smaller guide catheters 100, which may be more useful or necessary for certain procedural applications.

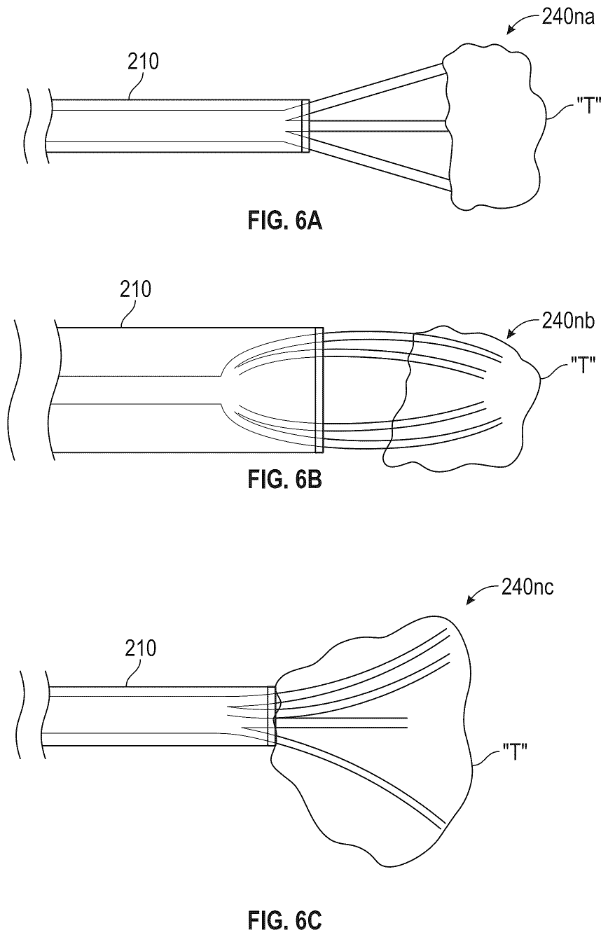

FIGS. 6A-6C illustrate various aspects of needle tines that may be usable with the intratumoral injection instrument 200 (with or without the use of a deflector 230). Each of the plurality of needle tines 240na (FIG. 6A), 240nb (FIG. 6B), and 240nc (FIG. 6C) are self-expanding and possess a preset curvature that changes the deployment angle of the plurality of needle tines 240na, 240nb, 240nc as they are deployed from the sleeve 210 and/or as they are inserted into a target. In particular, each of the plurality of needle tines 240na, 240nb, 240nc may be usable with an intratumoral injection instrument 200 that does not include a deflector 230 as a deflector 230 is not necessary to change the deployment angle of the plurality of needle tines 240na, 240nb, 240nc.

FIG. 6A illustrates a plurality of needle tines 240na having a hypo-fork configuration. Each needle tine of the plurality of needle tines 240na splay outward upon exiting the sleeve 210. Additionally, the plurality of needle tines 240na could splay even further out upon engagement with the tumor “T” and upon further penetration into the tumor “T”. FIG. 6B illustrates a plurality of needle tines 240nb having a distal-foci configuration. Each needle tine of the plurality of needle tines 240nb splay out promptly upon exiting the sleeve 210 and then curve inward upon engagement and interaction with the tumor “T”. FIG. 6C illustrates a plurality of needle tines 240nc having a distal expansion configuration. The plurality of needle tines 240nc do not splay outward upon exiting the sleeve 210. Rather, the plurality of needle tines 240nc only splay outward upon insertion into the tumor “T”. The configuration illustrated in FIG. 6C is particularly useful in applications that require intratumoral injection of the therapeutic agent into the peripheral boundaries of the tumor “T”. It should be appreciated that each of the plurality of needle tines 240na, 240nb, 240nc may be configured to splay in a single plane and/or to splay in multiple planes (e.g., in a three-dimensional configuration) upon insertion into the tumor “T”.

The intratumoral injection instrument 200 is illustrated as a manually actuatable surgical instrument, but it is appreciated that the intratumoral injection instrument 200 may be an electrically powered surgical instrument including an electrically powered handle assembly that may support one or more batteries (not shown). It is envisioned that the disclosed aspects could also be incorporated into a surgical instrument that is configured for use with a robotic system that does not include a handle assembly, or to a surgical instrument including a manually actuated handle assembly.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. Also, one skilled in the art will appreciate further features and advantages of the disclosure based on the above-described embodiments. Accordingly, the disclosure is not to be limited by what has been particularly shown and described, except as indicated by the appended claims.

What is claimed is:

1. An injection instrument comprising:
a supply chamber configured to store a therapeutic agent;
a handle assembly;
a sleeve extending from the handle assembly and defining a lumen;
a distal ring disposed at a distal end of the sleeve;
a plurality of needle tines extending through the lumen of the sleeve and movable through the lumen of the sleeve via the handle assembly, wherein the plurality of needle tines is configured to dispense the therapeutic agent from the supply chamber; and
a deflector extending through the lumen of the sleeve and movable through the lumen of the sleeve via the handle assembly, wherein retraction of the deflector relative to the sleeve and the plurality of needle tines causes the plurality of needle tines to deflect away from a central longitudinal axis of the sleeve against the distal ring to change an angle between a trajectory of the plurality of needle tines and the central longitudinal axis.

2. The injection instrument of claim 1, wherein the deflector includes an anvil portion configured to abut the plurality of needle tines and apply a force to the plurality of needle tines against the distal ring.

3. The injection instrument of claim 1, wherein:
distal advancement of the plurality of needle tines relative to the deflector a first distance deflects the plurality of needle tines at a first angle relative to the central longitudinal axis; and
distal advancement of the plurality of needle tines relative to the deflector a second distance deflects the plurality of needle tines at a second angle relative to the central longitudinal axis.

4. The injection instrument of claim 1, wherein:
retraction of the deflector relative to the sleeve and the plurality of needle tines a first distance deflects the plurality of needle tines at a first angle relative to the central longitudinal axis; and
retraction of the deflector relative to the sleeve and the plurality of needle tines a second distance deflects the plurality of needle tines at a second angle relative to the central longitudinal axis.

5. The injection instrument of claim 1, wherein a plurality of rounded grooves is defined along a length of an outer surface of the deflector and each needle tine of the plurality of needle tines is disposed within a respective rounded groove of the plurality of rounded grooves.

6. The injection instrument of claim 1, wherein the plurality of needle tines is formed of a shape memory material possessing a predefined curvature.

7. The injection instrument of claim 1, wherein a distal end of the deflector includes a blunt dilator tip.

8. The injection instrument of claim 1, wherein the handle assembly includes:
a first handle operably coupled to the plurality of needle tines and configured to control movement of the needle tines relative to the deflector;
a second handle operably coupled to the sleeve and configured to control movement of the sleeve relative to the plurality of needle tines and the deflector; and
a third handle operably coupled to the deflector and configured to control movement of the deflector relative to the plurality of needle tines and the sleeve.

9. The injection instrument of claim 1, wherein the sleeve is formed of a flexible material and the distal ring is formed of a rigid material.

10. The injection instrument of claim 1, wherein each needle tine of the plurality of needle tines is independently movable relative to other needle tines of the plurality of needle tines.

11. A surgical intratumoral injection system comprising:
a guide catheter defining a lumen and configured to be navigated to a target; and
a surgical intratumoral injection instrument configured to be inserted through the lumen of the guide catheter to access the target, the surgical intratumoral injection instrument including:
a supply chamber configured to store a therapeutic agent;
a handle assembly;
a sleeve extending from the handle assembly and defining a lumen;
a distal ring disposed at a distal end of the sleeve;
a plurality of needle tines extending through the lumen of the sleeve and movable through the lumen of the sleeve via the handle assembly, wherein the plurality of needle tines is configured to dispense the therapeutic agent from the supply chamber; and
a deflector extending through the lumen of the sleeve and movable through the lumen of the sleeve via the handle assembly, wherein retraction of the deflector relative to the sleeve and the plurality of needle tines causes the plurality of needle tines to deflect away from a central longitudinal axis of the sleeve against the distal ring to change an angle between a trajectory of the plurality of needle tines and the central longitudinal axis.

12. The surgical intratumoral injection system of claim 11, wherein the deflector includes an anvil portion configured to abut the plurality of needle tines and apply a force to the plurality of needle tines against the distal ring.

13. The surgical intratumoral injection system of claim 11, wherein:
distal advancement of the plurality of needle tines relative to the deflector a first distance deflects the plurality of needle tines at a first angle relative to the central longitudinal axis; and
distal advancement of the plurality of needle tines relative to the deflector a second distance deflects the plurality of needle tines at a second angle relative to the central longitudinal axis.

14. The surgical intratumoral injection system of claim 11, wherein:
retraction of the deflector relative to the sleeve and the plurality of needle tines a first distance deflects the plurality of needle tines at a first angle relative to the central longitudinal axis; and
retraction of the deflector relative to the sleeve and the plurality of needle tines a second distance deflects the plurality of needle tines at a second angle relative to the central longitudinal axis.

15. The surgical intratumoral injection system of claim 11, wherein a plurality of rounded grooves is defined along a length of an outer surface of the deflector and each needle tine of the plurality of needle tines is disposed within a respective rounded groove of the plurality of rounded grooves.

16. The surgical intratumoral injection system of claim 11, wherein the plurality of needle tines is formed of a shape memory material possessing a predefined curvature.

17. The surgical intratumoral injection system of claim 11, wherein a distal end of the deflector includes a blunt dilator tip.

18. The surgical intratumoral injection system of claim 11, wherein the handle assembly includes:
a first handle operably coupled to the plurality of needle tines and configured to control movement of the needle tines relative to the deflector;
a second handle operably coupled to the sleeve and configured to control movement of the sleeve relative to the plurality of needle tines and the deflector; and
a third handle operably coupled to the deflector and configured to control movement of the deflector relative to the plurality of needle tines and the sleeve.

19. The surgical intratumoral injection system of claim 11, wherein the sleeve is formed of a flexible material and the distal ring is formed of a rigid material.

20. A surgical intratumoral injection instrument comprising:
a supply chamber configured to store a therapeutic agent;
a handle assembly;
a sleeve extending from the handle assembly and defining a lumen; and
a plurality of needle tines extending through the lumen of the sleeve and movable through the lumen of the sleeve via the handle assembly, wherein the plurality of needle tines is configured to dispense the therapeutic agent from the supply chamber to a tumor, and wherein the plurality of needle tines is formed of a shape memory material configured to splay radially outward from a central longitudinal axis of the sleeve when the plurality of needle tines is deployed from the sleeve and configured to curve inward toward the central longitudinal axis of the sleeve as the plurality of needle tines is inserted into the tumor.
